(19) United States
(12) Patent Application Publication
(10) Pub. No.: US 2026/0091222 A1
(43) Pub. Date: Apr. 2, 2026

(54) Devices, Systems, and Methods for Aligning and Attaching Components of an Electrode Assembly

(71) Applicant: Novocure GmbH, Baar (CH)

(72) Inventors: Stas Obuchovsky, Haifa (IL); Yoram Wasserman, Haifa (IL); David Shapiro, Haifa (IL); Amir Ariel, Haifa (IL)

(21) Appl. No.: 19/343,840

(22) Filed: Sep. 29, 2025

Related U.S. Application Data
(60) Provisional application No. 63/701,247, filed on Sep. 30, 2024.

(51) Int. Cl. A61N 1/04; A61N 1/36

(52) U.S. Cl. CPC A61N 1/0496; A61N 1/0408; A61N 1/36002

(57) ABSTRACT

An assembly for providing tumor treating fields includes an electrode subassembly with at least one electrode element. The electrode subassembly has a skin-facing surface and an opposing outwardly facing surface. The assembly further includes a skin contact subassembly removably coupled to the skin-facing surface of the electrode subassembly. The skin contact subassembly includes a base portion. The base portion includes an electrically conductive skin contact layer configured to contact skin of a subject. The skin contact subassembly further includes a plurality of flaps, each flap having a respective edge congruent with or attached to the base portion and pivotably coupled to the base portion. The respective edges of the plurality of flaps cooperate to at least partially surround a receiving area. The electrode subassembly has an outer perimeter that overlies the receiving area and fits within an outer perimeter of the receiving area.

### CROSS-REFERENCE TO RELATED APPLICATION

This application claims priority to and the benefit of the filing date of U.S. Provisional Patent Application No. 63/701,247, filed September 30, 2024, which is hereby incorporated herein by reference in its entirety.

### BACKGROUND

Tumor Treating Fields (TTFields) therapy is a proven approach for treating tumors using alternating electric fields at frequencies between 50 KHz – 1 MHz, such as 100-500 kHz. The alternating electric fields are induced by electrode assemblies (e.g., arrays of capacitively coupled electrodes, also called transducer arrays) placed on opposite sides of a target location in the subject’s body. When an AC voltage is applied between opposing electrode assemblies, an AC current is coupled through the electrode assemblies and into the subject’s body. And higher currents are strongly correlated with higher efficacy of treatment.

Conventionally, the electrode assemblies used during application of TTFields typically include an electrically conductive hydrogel layer that serves as a skin contact layer that adheres to the skin of the subject. The electrically conductive hydrogel typically has a shorter lifespan than the rest of the electrode assembly. For example, with use, the skin contact layer can degrade, for example, by collecting oil and dirt, thereby reducing effectiveness of the electrically conductive hydrogel layer. The hydrogel is typically integral to the electrode assembly. Thus, upon expiration or contamination of the hydrogel, the entire electrode assembly must be disposed of and replaced.

### SUMMARY

Disclosed herein, in various aspects, are apparatuses and kits for applying TTFields. The apparatuses and kits can include an electrode subassembly and a skin contact subassembly that is configured to be removably coupled to the electrode subassembly. The apparatuses and kits disclosed herein can additionally facilitate alignment and/or attachment between the electrode subassembly and the skin contact subassembly.

In one aspect, an apparatus comprises an electrode subassembly, the electrode subassembly comprising at least one electrode element, the electrode subassembly having a skin-facing surface and an opposing outwardly facing surface. The apparatus further comprises a skin contact subassembly removably coupled to the skin-facing surface of the electrode subassembly. The skin contact subassembly comprises a base portion extending along a first axis and a second axis that is perpendicular to the first axis. The base portion comprises an electrically conductive skin contact layer configured to contact skin of a subject. The skin contact subassembly further comprises a plurality of flaps, each flap having a respective edge congruent with or attached to the base portion and pivotably coupled to the base portion. The respective edges of the plurality of flaps cooperate to at least partially surround a receiving area of the base portion configured to receive at least a portion of the electrode subassembly thereagainst. The electrode subassembly has an outer perimeter that overlies the receiving area and fits within an outer perimeter of the receiving area.

In another aspect, a kit and a method of using the kit are disclosed. The kit includes an electrode subassembly comprising at least one electrode element and having a skin-facing surface, an opposing outwardly facing surface, and an outer perimeter. The kit further includes one or more skin contact subassemblies each of which is configured to be removably coupled to the skin-facing surface of the electrode subassembly. Each skin contact subassembly of the one or more skin contact subassemblies comprises a base portion extending along a first axis and a second axis that is perpendicular to the first axis, wherein the base portion comprises an electrically conductive skin contact layer configured to contact skin of a subject. Each skin contact subassembly further comprises a plurality of flaps each flap having a respective edge congruent with or attached to the base portion and pivotably coupled to the base portion, wherein the respective edges of the plurality of flaps cooperate to at least partially surround a receiving area of the base portion configured to receive at least a portion of the electrode subassembly thereagainst. Each skin contact subassembly of the one or more skin contact subassemblies is configured to receive the electrode subassembly thereagainst so that an entirety of the outer perimeter of the electrode subassembly overlies the receiving area and fits within an outer perimeter of the receiving area. The method comprises positioning the electrode subassembly against the receiving area of a skin contact subassembly of the one or more skin contact subassemblies so that the entirety of the outer perimeter of the electrode subassembly overlies the receiving area of the skin contact subassembly and fits within an outer perimeter of the receiving area. The flaps can be folded over the outwardly facing surface of the electrode subassembly.

In one aspect, an assembly includes an electrode subassembly comprising at least one electrode element and having a skin-facing surface and an opposing outwardly facing surface. A skin contact subassembly is removably coupled to the skin-facing surface of the electrode subassembly. The skin contact subassembly comprises a base portion extending along a first axis and a second axis that is perpendicular to the first axis. The base portion comprises an electrically conductive skin contact layer configured to contact skin of a subject. The skin contact subassembly further comprises one or more flaps, with each flap having a respective edge congruent with or attached to the base portion and pivotably coupled to an edge of the base portion. Each flap of the one or more flaps is configured to pivot about its respective edge to attach to the outwardly facing surface of the electrode subassembly.

In one aspect, a kit includes an electrode subassembly comprising at least one electrode element and having a skin-facing surface, and at least one peripheral edge. The kit further includes a skin contact subassembly that is configured to be removably coupled to the skin-facing surface of the electrode subassembly. The skin contact subassembly has a skin-facing surface and at least one peripheral edge. The kit further includes a jig that is configured to contact portions of one or both of the electrode subassembly and the skin contact subassembly to orient the electrode subassembly and the skin contact subassembly relative to each other along a first axis and a second axis that is perpendicular to the first axis.

Systems and methods for using the disclosed apparatuses and kits (e.g., treatment assemblies) are also disclosed.

### BRIEF DESCRIPTION OF THE DRAWINGS

FIG. 1 is a schematic top view representation of an exemplary treatment assembly as disclosed herein.

FIG. 2 is a schematic cross-sectional representations of exemplary treatment assemblies as disclosed herein, taken in the plane 2-2’ of FIG. 1, according to various non-limiting embodiments.

FIG. 3 is a perspective view of an exemplary embodiment of an electrode assembly as disclosed herein.

FIG. 4 is an exploded view of an exemplary embodiment of an electrode assembly and a jig as disclosed herein.

FIG. 5 is an exploded view of another exemplary embodiment of an electrode assembly and a jig as disclosed herein.

FIG. 6A is a top plan view of a skin contact subassembly. FIG. 6B is an exploded side view of an exemplary electrode assembly.

FIG. 7 is a perspective view of a system for securing an electrode assembly to a patient as disclosed herein.

FIG. 8 is a perspective view of an exemplary embodiment of an electrode assembly as disclosed herein.

Various embodiments are described in detail below with reference to the accompanying drawings, wherein like reference numerals represent like elements.

### DETAILED DESCRIPTION

This application describes exemplary electrode assemblies that can be used, e.g., for delivering TTFields to a subject’s body and treating one or more cancers or tumors located in the subject’s body.

The present invention can be understood more readily by reference to the following detailed description, examples, drawings, and claims, and their previous and following description. However, it is to be understood that this invention is not limited to the specific apparatuses, devices, systems, and/or methods disclosed unless otherwise specified, and as such, of course, can vary.

Headings are provided for convenience only and are not to be construed to limit the invention in any manner. Embodiments illustrated under any heading or in any portion of the disclosure may be combined with embodiments illustrated under the same or any other heading or other portion of the disclosure.

Any combination of the elements described herein in all possible variations thereof is encompassed by the invention unless otherwise indicated herein or otherwise clearly contradicted by context.

As used in the specification and the appended claims, the singular forms “a,” “an” and “the” include plural referents unless the context clearly dictates otherwise. Thus, it is contemplated that disclosure of a singular form of an element can provide support for embodiments in which only a single such element is provided, as well as support for embodiments in which a plurality of such elements are provided.

As used herein, the term “conductive adhesive or gel” should be understood to mean “conductive adhesive or conductive gel.” Further, the term “conductive gel” should be understood to include hydrogel.

As used herein, the term “front” refers to a direction or position toward the skin, and “rear” refers to a direction or position away from or opposite the skin.

Existing electrode assemblies for providing TTFields are unitarily constructed as an assembly with one or more electrode elements and a skin contact layer. As noted above, the whole construct needs to be replaced once the skin contact layer has been contaminated or has degraded. Disclosed herein are electrode assemblies that permit replacement of the skin contact layer and subsequent reuse of the electrode assembly (i.e. a 2-part array comprising an electrode subassembly and a skin contact subassembly).

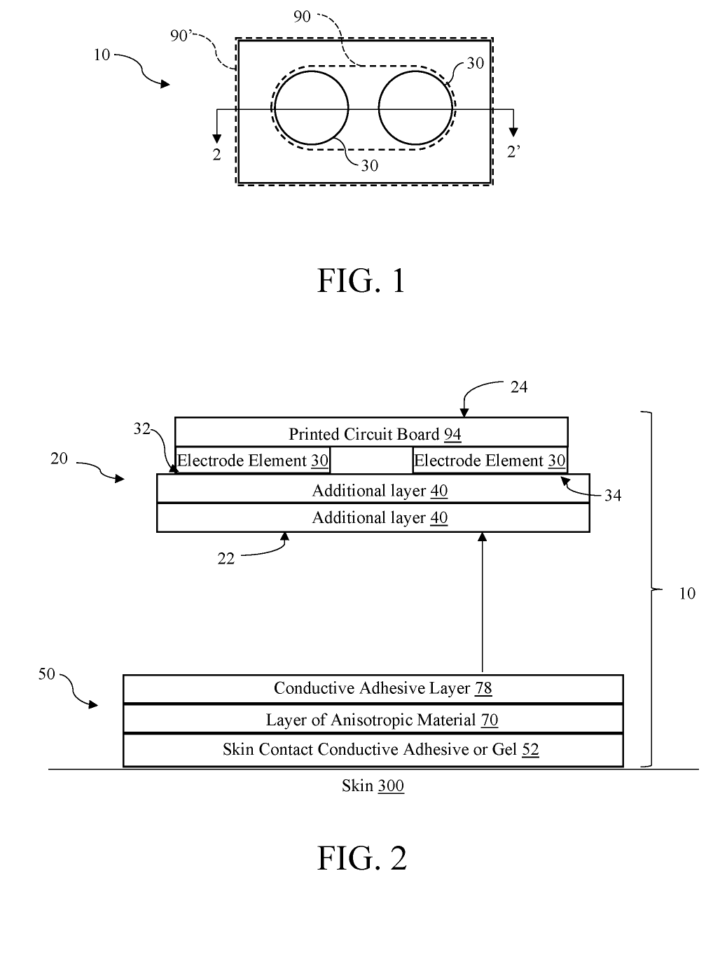

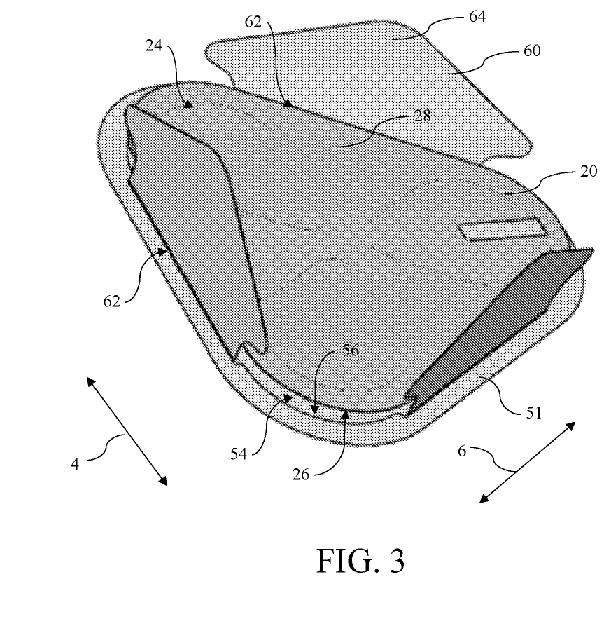

FIG. 1 illustrates a top schematic view of an apparatus 10 for providing TTFields and shows two electrode elements 30 (although the apparatus could have one, two or more than two electrode elements 30). In some optional aspects, the apparatus 10 can comprise a plurality of electrode elements 30. In these aspects, the electrode elements 30 can be wired together (e.g., using wires, or traces on a printed circuit board 94 that can optionally be a flex circuit, etc.). As discussed further herein, the apparatus 10 can have an operative conductive area. For example, as shown in FIG. 1, the operative conductive area 90 can be defined by an outer perimeter that surrounds every electrode of the at least one electrode element 30 (e.g., an areal footprint of the at least one electrode element). Also discussed herein, in alternative aspects in which the apparatus comprises a layer of anisotropic conductive material 70 (e.g., FIG. 2), the operative conductive area 90’ can be defined by the outer perimeter of the anisotropic material.

Referring also to FIG. 2, showing a schematic cross-sectional representation of the apparatus 10, taken in the plane 2-2’, the apparatus 10 can comprise an electrode subassembly 20, the electrode subassembly 20 comprising at least one electrode element 30 having a skin-facing side 32 and a skin-facing surface 34. The electrode subassembly 20 can have a skin-facing surface 22 and an opposing outwardly facing surface 24. In some optional aspects, the at least one electrode element 30 can define the skin-facing surface 22 of the electrode subassembly 20.

In other aspects, the electrode assembly 20 can comprise one or more layers 40 on the skin-facing side of the at least one electrode element 30, and one of the one or more layers 40 can define the skin-facing surface 22 of the electrode subassembly 20. The one or more layers 40 can include, for example, one or more of: a layer of anisotropic material, a conductive adhesive layer, a polymer layer that is electrically conductive or a dielectric layer. In some aspects, the dielectric layer can comprise a ceramic. In some aspects, the dielectric layer can comprise a dielectric polymer. In exemplary aspects the dielectric layer can have a dielectric constant greater than 10 or greater than 20. In various aspects, the dielectric layer can be embodied as separate discrete (discontinuous) layers on the surface of each electrode or a single (continuous) layer covering a plurality of electrode elements or covering all of the electrode elements. In some aspects, the electrode subassembly 20 does not include a dielectric layer. In other aspects, the electrode subassembly 20 can be free of additional layers 40.

Referring to FIGS. 2-3, the apparatus 10 can further comprise a skin contact subassembly 50 removably coupled to the skin-facing surface 22 of the electrode subassembly 20. The skin contact subassembly 50 can comprise a base portion 51 extending along a first axis 4 and a second axis 6 that is perpendicular to the first axis (FIG. 3). The base portion 51 can comprise a skin contact conductive adhesive or gel 52 configured to contact skin 300 (FIG. 2) of a subject. For example, the skin contact conductive adhesive or gel 52 can be or comprise an acrylic or silicone polymer having conductive particles dispersed therein, such as, for example, metal particles or carbon particles. Alternatively, the skin contact conductive adhesive or gel 52 can be or comprise a hydrogel. In alternative embodiments, the base portion 51 can comprise a non-adhesive conductive skin contact layer configured to contact skin 300 of a subject. For example, the base portion 51 of the skin contact subassembly may comprise a layer of conductive silicone elastomer, wherein the layer of conductive silicone elastomer has a non-adhesive front face that is textured in a manner that makes the front face of the layer of conductive silicone adhere to human skin. Non-adhesive conductive silicone skin-contact material may be available as ElectroSkin Gecko product (available from Nanoleq, 8153 Rümlang, CH). The skin contact subassembly 50 can further comprise at least one flap 60 (optionally, a plurality of flaps 60). Each flap 60 can have a respective edge 62. Optionally, each edge 62 can be congruent with, or attached to, the base portion 51. More generally, each flap can pivotably couple to the base portion 51 at its respective edge 62. Each flap 60 can at least partially surround a receiving area 54 of the base portion 51 that is configured to receive at least a portion of the electrode subassembly 20 thereagainst. In FIG. 3, the receiving area 54 is mostly obscured by the electrode subassembly 20; in use, when the electrode subassembly 20 and the skin contact subassembly 50 are properly aligned, the receiving area 54 may be completely or almost completely obscured. When a plurality of flaps are provided, the respective edges 62 of the plurality of flaps 60 can cooperate to at least partially surround the receiving area 54 of the base portion 51. In this way, the flap(s) 60 can constrain positioning of the electrode subassembly 20 relative to the base portion 51 along the first and second axes 4, 6. Accordingly, the electrode subassembly 20 can have an outer perimeter 26 that overlies the receiving area. The outer perimeter 26 of the electrode subassembly 20 can fit within an outer perimeter 56 of the receiving area 54 or can fit within a subset area of the outer perimeter 56 of the receiving area 54.

Generally herein, the skin-facing surface of the skin contact conductive adhesive or gel 52 defines a skin-facing surface of the skin contact subassembly 50. The skin contact conductive adhesive or gel 52 can be electrically coupled to the at least one electrode element 30 when the skin contact subassembly 50 is disposed against the skin-facing surface 22 of the electrode subassembly 20. Optionally, in these aspects, the skin-facing surface 22 of the electrode subassembly 20 can be formed by the skin-facing surface of the electrically conductive additional layer 40 of the electrode subassembly 20. In exemplary, optional aspects, the skin contact conductive adhesive or gel 52 can comprise hydrogel. In other aspects, the skin contact conductive adhesive or gel 52 can be free of hydrogel. For example, in some optional aspects, the skin contact conductive adhesive or gel 52 can comprise a conductive adhesive composite (described further herein). The conductive adhesive composite can comprise conductive particles dispersed therethrough. For example, the conductive particles can comprise carbon. In exemplary aspects, the conductive particles can comprise one or more of: graphite powder, carbon flakes, carbon fibers, carbon granules, carbon nanotubes, carbon nanowires, carbon black powder, or carbon microcoils. In some aspects, the conductive particles can comprise metal.

The skin contact subassembly 50 can further comprise one or more additional layers, such as, for example, and without limitation, a conductive adhesive layer 78 and/or a layer of anisotropic material 70.

In some aspects, the flap(s) 60 can be configured to pivot about their respective edges 62 to attach to the outwardly facing surface 24 of the electrode subassembly 20. In further aspects, the flap(s) 60 can each comprise adhesive or hook or loop material 64 that is configured to attach to the outwardly facing surface 24 of the electrode subassembly 20. In still further aspects, the flap(s) 60 can be configured to attach to adhesive or hook or loop material 28 on the outwardly facing surface of the electrode subassembly. In further aspects, both the flap(s) 60 and the outwardly facing surface 24 of the electrode subassembly 20 can each comprise adhesive or hook or loop material 64 that is configured to attach the respective surfaces. In other aspects, the flap(s) 60 can each comprise a magnet or ferromagnetic material that is configured to attach to a corresponding magnet or ferromagnetic material of the electrode subassembly 20 (not shown). Optionally, the corresponding magnet or ferromagnetic material can be on the outwardly facing surface of the electrode subassembly 20.

In some aspects, an entirety of the operative conductive area 90 or 90’ can overlie the receiving area 54 surrounded by the flap(s) 60 of the skin contact subassembly 50. In still further aspects, the entirety of the operative conductive area 90 or 90’ can overlie an anisotropic layer of either the skin contact subassembly 50 or the electrode subassembly 20. In this way, by using the flap(s) 60 to align the electrode subassembly 20 and the skin contact subassembly 50, electrical shorts to the skin of the subject can be avoided. More generally, it is contemplated that receipt of the outer perimeter 26 of the electrode subassembly 20 against the receiving area 54, and particularly the outer perimeter 56 of the receiving area 54, corresponds to proper alignment between the electrode subassembly 20 and the skin contact subassembly 50.

In some aspects, the skin contact subassembly 50 can comprise at least three flaps 60. In further aspects, the skin contact subassembly 50 can comprise exactly three flaps 60, four flaps, five flaps, six flaps, or more flaps. In other aspects, the skin contact subassembly 50 can have only a single flap 60 or exactly two flaps. Accordingly, in various embodiments, the flap(s) 60 can fully or partially constrain the electrode subassembly 20 relative to the skin contact subassembly 50 along the first and second axes 4, 6.

In some aspects, a kit can comprise an electrode subassembly 20 and one or more skin contact subassemblies 50 each of which can be configured to be removably coupled to the skin-facing surface 22 of the electrode subassembly 20. Each skin contact subassembly 50 of the plurality of skin contact subassemblies can be configured to receive the electrode subassembly 20 thereagainst so that an entirety of the outer perimeter 26 of the electrode subassembly 20 overlies the receiving area 54 and fits within an outer perimeter 56 of the receiving area 54 or fits within a subset area of the outer perimeter 56 of the receiving area 54.

A method of using the kit can include positioning the electrode subassembly 20 against the receiving area 54 of a skin contact subassembly 50 so that the entirety of the outer perimeter 26 of the electrode subassembly overlies the receiving area of the skin contact subassembly and fits within or at least partially within an outer perimeter 56 of the receiving area 54 or fits within a subset area of the outer perimeter 56 of the receiving area 54. The flap(s) 60 can be folded over the outwardly facing surface of the electrode subassembly.

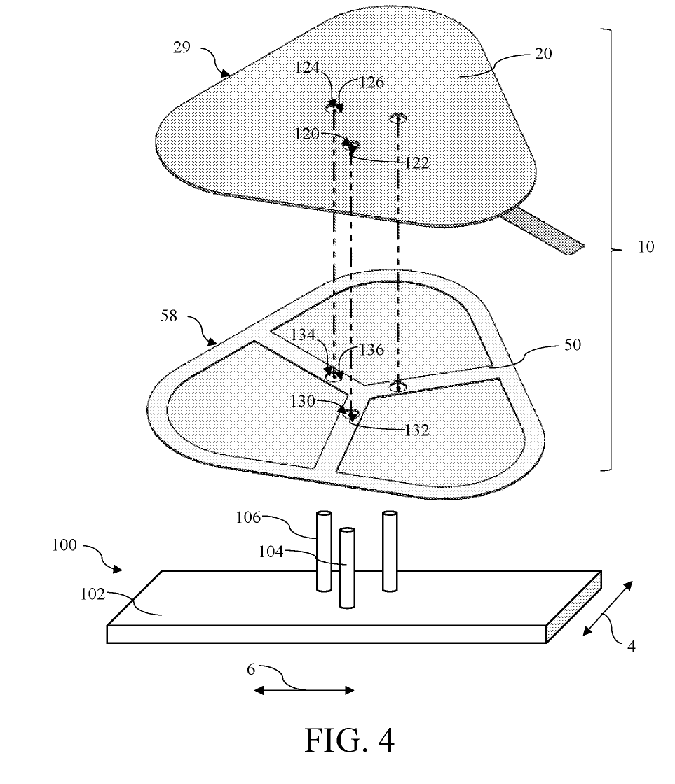

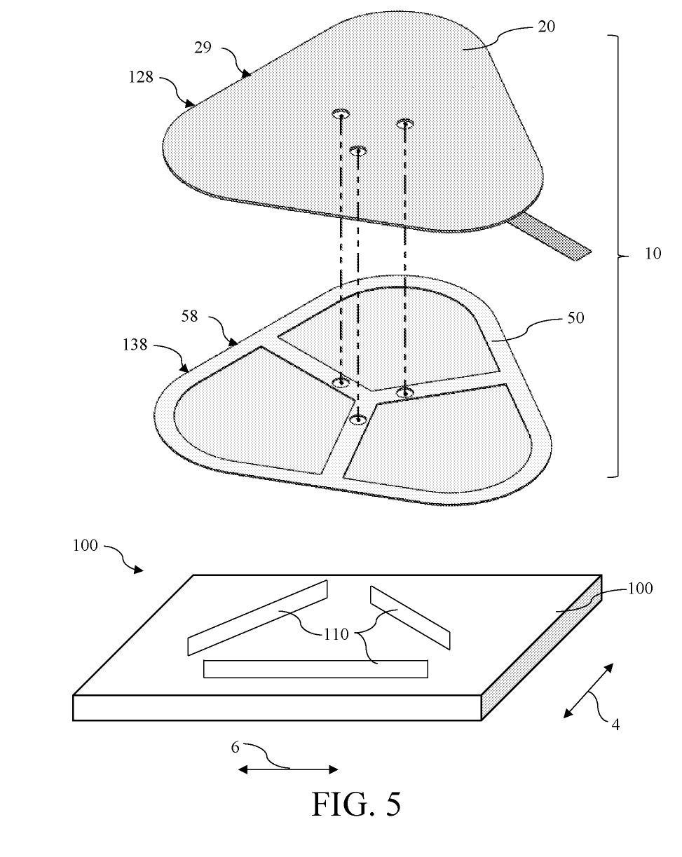

Referring to FIGS. 4-5, in some embodiments, a kit can include a jig 100 configured to align an electrode subassembly 20 and a skin contact subassembly 50. The electrode subassembly 20 can comprise at least one electrode element (FIGS. 1-2). The electrode assembly 20 can have a skin-facing surface 22 and at least one peripheral edge 29. The skin contact subassembly 50 can be configured to be removably coupled to the skin-facing surface 22 of the electrode subassembly 20. The skin contact subassembly can comprise a skin contact conductive adhesive or gel 52 (FIG. 2) or a non-adhesive conductive skin contact layer. The skin contact subassembly 50 can have at least one peripheral edge 58.

The jig 100 can be configured to contact portions of one or both of the electrode subassembly 20 and the skin contact subassembly 50 to orient the electrode subassembly and the skin contact subassembly relative to each other along a first axis 4 and a second axis 6 that is perpendicular to the first axis. For example, the jig 100 can be configured to contact portions of the peripheral edges of each of the electrode subassembly 20 and the skin contact subassembly 50. The at least one peripheral edge 29 of the electrode subassembly 20 that the jig 100 contacts can include an outer edge and, optionally, one or more inner edges. Similarly, the at least one peripheral edge 58 of the skin contact subassembly 50 that the jig contacts can include an outer edge and, optionally, one or more inner edges.

Referring to FIG. 4, in some aspects, the electrode subassembly 20 can include a first hole 120 having a first inner peripheral edge 122 surrounding the first hole of the electrode subassembly 20. The at least one peripheral edge 29 of the electrode subassembly 20 can comprise or encompass the first inner peripheral edge 122 of the first hole 120 of the electrode subassembly 20. The skin contact subassembly 50 can include a first hole 130 having a first inner peripheral edge 132 surrounding the first hole of the skin contact subassembly 50. The at least one peripheral edge 58 of the skin contact subassembly 50 can comprise or encompass the first inner peripheral edge 132 of the first hole 130 of the skin contact subassembly. The jig 100 can comprise a base 102 and a first protrusion 104 extending from the base 102. The first protrusion 104 can be configured to extend through the first hole 130 of the skin contact subassembly 50 and the first hole 120 of the electrode subassembly 20. The first protrusion 104 can contact the first inner peripheral edge 132 of the first hole 130 of the skin contact subassembly 50 and the first inner peripheral edge 122 of the first hole 120 of the electrode subassembly 20. In this way, receipt of the first protrusion 104 through the first holes of each of the electrode subassembly 20 and the skin contact subassembly 50 can at least partially align the electrode subassembly 20 and the skin contact subassembly 50.

In further aspects, the electrode subassembly 20 can further include a second hole 124 having a second inner peripheral edge 126 surrounding the second hole of the electrode subassembly. The at least one peripheral edge 29 of the electrode subassembly can comprise or encompass the second inner peripheral edge 126 of the second hole 124. The skin contact subassembly 50 can include a second hole 134 having a second inner peripheral edge 136 surrounding the second hole of the skin contact subassembly 50. The at least one peripheral edge 58 of the skin contact subassembly 50 can comprise or encompass the second inner peripheral edge 136 of the second hole 134 of the skin contact subassembly 50. The jig 100 can further comprises a second protrusion 106 extending from the base 102. The second protrusion 106 can be configured to extend through the second hole 134 of the skin contact subassembly 50 and the second hole 124 of the electrode subassembly 20 and contact the second inner peripheral edge 136 of the second hole 134 of the skin contact subassembly 50 and the second inner peripheral edge 126 of the second hole 124 of the electrode subassembly 20. In this way, receipt of both the first protrusion 104 through the first holes of each of the electrode subassembly 20 and the skin contact subassembly 50 and the second protrusion 106 through the second holes of each of the electrode subassembly 20 and the skin contact subassembly 50 can properly align the electrode subassembly 20 and the skin contact subassembly 50.

In further aspects, the jig 100 can comprise three or more protrusions. In these aspects, the electrode subassembly 20 and the skin contact subassembly 50 can include respective holes that receive each of the three or more protrusions.

Referring to FIG. 5, in some aspects, the at least one peripheral edge 29 of the electrode subassembly 20 can comprise an outer peripheral edge 128. The at least one peripheral edge 58 of the skin contact subassembly 50 can comprise an outer peripheral edge 138. The jig 100 can be configured to contact portions of the outer peripheral edges of each of the electrode subassembly 20 and the skin contact subassembly 50 to orient the electrode subassembly and the skin contact subassembly relative to each other. For example, the jig 100 can include sidewalls 110 that are configured to contact portions of the outer peripheral edge 128 of the electrode subassembly 20 as well as portions of the outer peripheral edge 138 of the skin contact subassembly 50 to align the electrode subassembly 20 and the skin contact subassembly 50 along the first and second axes 4, 6.

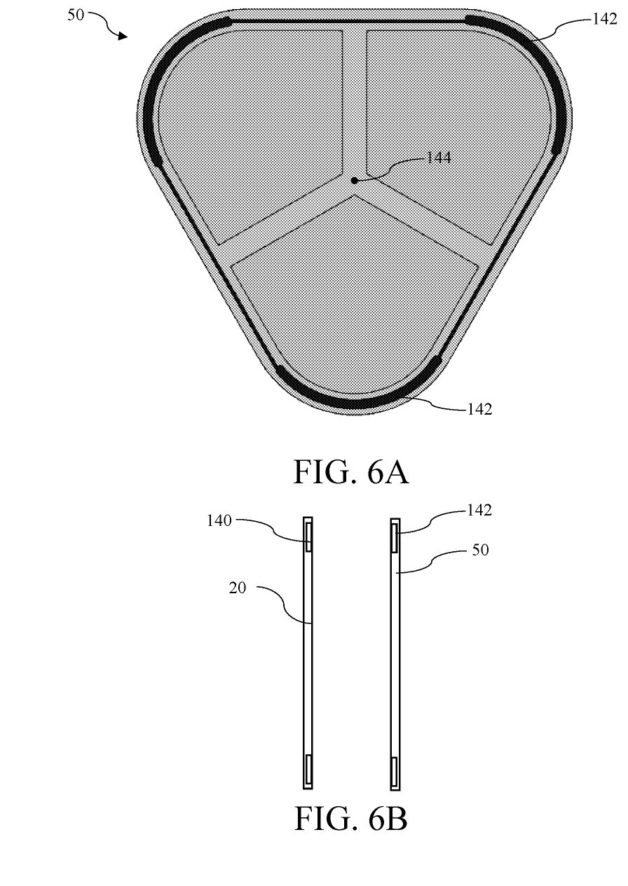

Referring to FIGS. 6A and 6B, in some aspects, one or more magnets or ferromagnetic materials can be used to align the electrode subassembly 20 and each skin contact subassembly 50. For example, a kit can comprise an electrode subassembly 20 and one or more skin contact subassemblies 50. The electrode subassembly 20 can comprise at least one magnet or ferromagnetic material 140. Each skin contact subassembly 50 can comprise at least one magnet or ferromagnetic material 142 that is positioned so that the at least one magnet or ferromagnetic material 142 is configured to magnetically attract the at least one magnet or ferromagnetic material 140 of the electrode subassembly 20 in a predetermined orientation between the electrode subassembly 20 and said skin contact subassembly 50. It is further contemplated that the electrode subassembly 20 can comprise a ferromagnetic material, and the skin contact subassembly 50 can comprise a magnet, and vice-versa.

The electrode subassembly 20 can have a centroid 144. Optionally, in exemplary aspects, the centroid 144 can correspond to a geometric centroid. In some aspects, the at least one magnet or ferromagnetic material 140 can be positioned radially outwardly from the centroid. The skin contact subassembly 50 can comprise a corresponding magnet or ferromagnetic material 142 positioned to engage the at least one magnet or ferromagnetic material 140 upon proper alignment between the electrode subassembly 20 and said skin contact subassembly 50. Accordingly, in addition to securing the electrode subassembly 20 and said skin contact subassembly 50, the at least one magnet or ferromagnetic material can effect alignment between the electrode subassembly 20 and said skin contact subassembly 50. Optionally, the electrode subassembly 20 can comprise a plurality of magnets positioned radially outwardly from the centroid 144 and the skin contact subassembly 50 can comprise corresponding magnets / ferromagnetic materials 142 positioned to engage the plurality of magnets / ferromagnetic materials 140.

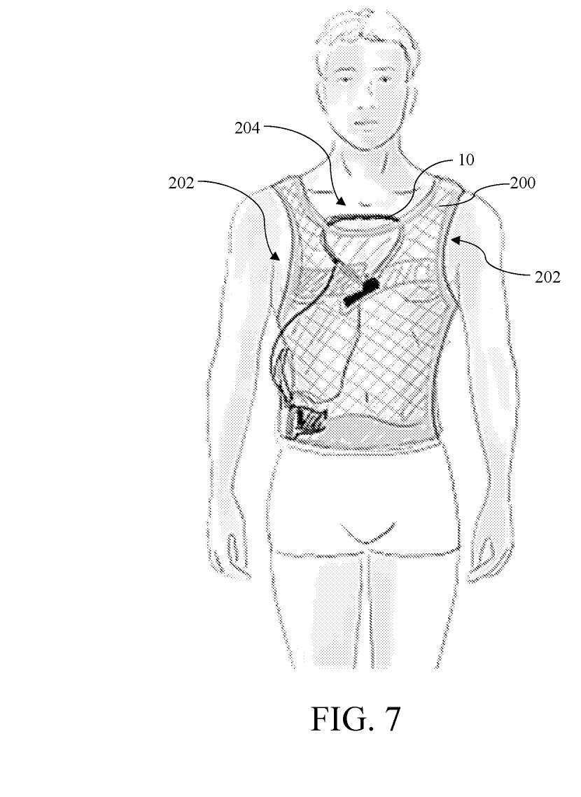

In some aspects, and with reference to FIG. 7, a kit can comprise a netting or elasticated band/garment 200 configured to be worn by a subject. For example, the netting or elasticated band/garment 200 can be configured to be worn on a torso of the subject. In these aspects, the torso can include at least one armhole 202 (e.g., a pair of armholes) and a head opening 204. The netting or elasticated band/garment can be configured to extend over an electrode assembly 10 so that the electrode assembly is positioned between the netting or elasticated band/garment and the subject. The netting or elasticated band/garment 200 can be configured to apply a pressure against the electrode assembly 10 to secure the electrode assembly against the subject.

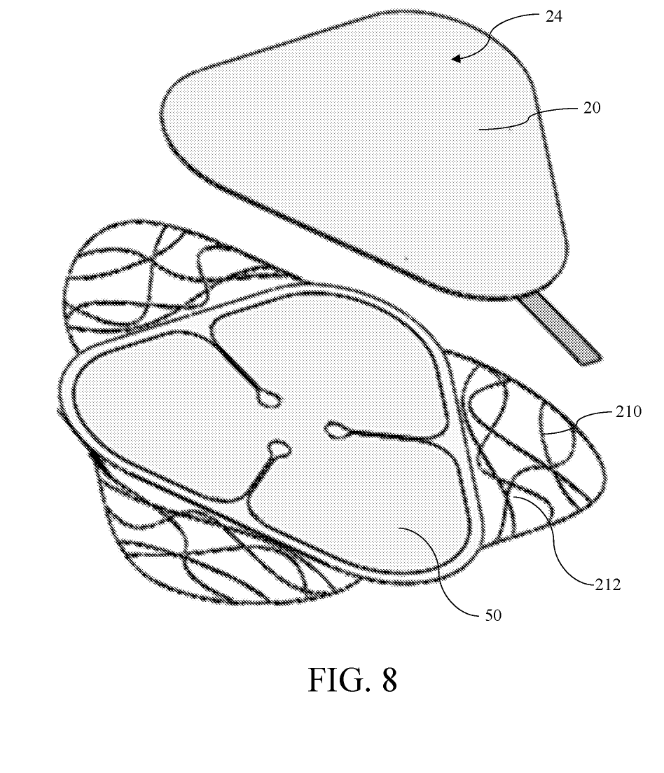

Referring also to FIG. 8, the skin contact subassembly 50 of the electrode assembly 10 can comprise an integral netting or elasticated band 210. The integral netting or elasticated band / garment or elasticated band / garment or elasticated band 210 can comprise one or more portions 212 that extend outwardly from a periphery of the electrically conductive skin contact layer and fold around the outwardly facing surface 24 of the electrode subassembly 20. An adhesive or hook or loop material can adhere the netting to the electrode subassembly 20.

In one aspect, a kit comprises an electrode subassembly 20 having at least one peripheral edge 26 (FIG. 3). The kit can further comprise one or more skin contact subassemblies 50 that are each configured to be removably coupled to the skin-facing surface 22 of the electrode subassembly 20. One of the electrode subassembly 20 or the skin contact subassembly 50 can comprise at least one marking that is configured to align with an edge of the other of the electrode subassembly 20 or the skin contact subassembly 50 upon alignment between the electrode subassembly or the skin contact subassembly. The at least one marking can comprise a first color. In other aspects, the at least one marking can comprise a first color and a second color that differs from the first color. The second color can be a color contrasting with the first color. Optionally, in these aspects, at least one of the first color or the second color can indicate misalignment. For example, exposure of the first color or the second color can be indicative of misalignment, whereas fully covering the first color or the second color can be indicative of alignment. In various aspects, the first color or the second color can be red, orange, yellow, green, blue, purple, black, or another color.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: An assembly comprising: an electrode subassembly comprising at least one electrode element and having a skin-facing surface and an opposing outwardly facing surface; and a skin contact subassembly removably coupled to the skin-facing surface of the electrode subassembly, wherein the skin contact subassembly comprises: a base portion extending along a first axis and a second axis that is perpendicular to the first axis, wherein the base portion comprises an electrically conductive skin contact layer configured to contact skin of a subject; a plurality of flaps, each flap having a respective edge congruent with or attached to the base portion and pivotably coupled to the base portion, wherein the respective edges of the plurality of flaps cooperate to at least partially surround a receiving area of the base portion configured to receive at least a portion of the electrode subassembly thereagainst, wherein the electrode subassembly has an outer perimeter that overlies the receiving area and fits within an outer perimeter of the receiving area.

Aspect 2: The assembly of aspect 1, wherein the plurality of flaps are configured to pivot about their respective edges to attach to the outwardly facing surface of the electrode subassembly.

Aspect 3: The assembly of aspect 2, wherein the plurality of flaps each comprise adhesive or hook or loop material that is configured to attach to the outwardly facing surface of the electrode subassembly.

Aspect 4: The assembly of aspect 2 or aspect 3, wherein the electrode subassembly comprises adhesive or hook or loop material on the outwardly facing surface of the electrode subassembly, and wherein the plurality of flaps are configured to attach to the adhesive or hook or loop material on the outwardly facing surface of the electrode subassembly.

Aspect 5: The assembly of any one of aspects 2-4, wherein the electrode subassembly comprises a magnet or ferromagnetic material on the outwardly facing surface of the electrode subassembly, and wherein the plurality of flaps each comprise a magnet or ferromagnetic material that is configured to attach to the magnet or ferromagnetic material on the outwardly facing surface of the electrode subassembly.

Aspect 6: The assembly of any one of the preceding aspects, wherein the electrode subassembly has an operative conductive area, wherein the operative conductive area overlies the receiving area surrounded by the plurality of flaps of the skin contact subassembly.

Aspect 7: The assembly of any one of the preceding aspects, wherein receipt of the outer perimeter of the electrode subassembly against the receiving area corresponds to proper alignment between the electrode subassembly and the skin contact subassembly.

Aspect 8: The assembly of any one of the preceding aspects, wherein the plurality of flaps of the skin contact subassembly comprises at least three flaps.

Aspect 9: A kit comprising: an electrode subassembly comprising at least one electrode element and having a skin-facing surface, an opposing outwardly facing surface, and an outer perimeter; and one or more skin contact subassemblies each of which is configured to be removably coupled to the skin-facing surface of the electrode subassembly, wherein each skin contact subassembly of the one or more skin contact subassemblies comprises: a base portion extending along a first axis and a second axis that is perpendicular to the first axis, wherein the base portion comprises an electrically conductive skin contact layer configured to contact skin of a subject; and a plurality of flaps each flap having a respective edge congruent with or attached to the base portion and pivotably coupled to the base portion, wherein the respective edges of the plurality of flaps cooperate to at least partially surround a receiving area of the base portion configured to receive at least a portion of the electrode subassembly thereagainst, wherein each skin contact subassembly of the one or more skin contact subassemblies is configured to receive the electrode subassembly thereagainst so that an entirety of the outer perimeter of the electrode subassembly overlies the receiving area and fits within an outer perimeter of the receiving area.

Aspect 10: A method of using the kit of aspect 9, the method comprising: positioning the electrode subassembly against the receiving area of a skin contact subassembly of the one or more skin contact subassemblies so that the entirety of the outer perimeter of the electrode subassembly overlies the receiving area of the skin contact subassembly and fits within an outer perimeter of the receiving area; and folding the flaps over the outwardly facing surface of the electrode subassembly.

Aspect 11: A kit comprising: an electrode subassembly comprising at least one electrode element and having a skin-facing surface, and at least one peripheral edge; a skin contact subassembly that is configured to be removably coupled to the skin-facing surface of the electrode subassembly, wherein the skin contact subassembly has a skin-facing surface and at least one peripheral edge; and a jig that is configured to contact portions of one or both of the electrode subassembly and the skin contact subassembly to orient the electrode subassembly and the skin contact subassembly relative to each other along a first axis and a second axis that is perpendicular to the first axis.

Aspect 12: The kit of aspect 11, wherein the electrode subassembly includes a first hole having a first inner peripheral edge surrounding the first hole of the electrode subassembly, wherein the at least one peripheral edge of the electrode subassembly encompasses the first inner peripheral edge of the first hole of the electrode subassembly, wherein the skin contact subassembly includes a first hole having a first inner peripheral edge surrounding the first hole of the skin contact subassembly, wherein the at least one peripheral edge of the skin contact subassembly encompasses the first inner peripheral edge of the first hole of the skin contact subassembly, wherein the jig comprises: a base; and a first protrusion extending from the base, wherein the first protrusion is configured to extend through the first hole of the electrode subassembly and the first hole of the skin contact subassembly and contact the first inner peripheral edge of the first hole of the electrode subassembly and the first inner peripheral edge of the first hole of the skin contact subassembly.

Aspect 13: The kit of aspect 12, wherein the electrode subassembly includes a second hole having a second inner peripheral edge surrounding the second hole of the electrode subassembly, wherein the at least one peripheral edge of the electrode subassembly encompasses the second inner peripheral edge of the second hole, wherein the skin contact subassembly includes a second hole, having a second inner peripheral edge surrounding the second hole of the skin contact subassembly wherein the at least one peripheral edge of the skin contact subassembly encompasses the second inner peripheral edge of the second hole of the skin contact subassembly, wherein the jig further comprises a second protrusion extending from the base, wherein the second protrusion is configured to extend through the second hole of the electrode subassembly and the second hole of the skin contact subassembly and contact the second inner peripheral edge of the second hole of the electrode subassembly and the second inner peripheral edge of the second hole of the skin contact subassembly.

Aspect 14: The kit of any one of aspects 11-13, wherein the at least one peripheral edge of the electrode subassembly comprises an outer peripheral edge, wherein the at least one peripheral edge of the skin contact subassembly comprises an outer peripheral edge, wherein the jig is configured to contact portions of the outer peripheral edges of each of the electrode subassembly and the skin contact subassembly to orient the electrode subassembly and the skin contact subassembly relative to each other.

Aspect 15: The kit of any one of aspects 11-14, wherein the jig is configured to contact portions of both of the electrode subassembly and the skin contact subassembly to orient the electrode subassembly and the skin contact subassembly relative to each other along the first axis and the second axis.

Aspect 16: A kit comprising: an electrode subassembly comprising: at least one electrode element having a skin-facing side; and at least one magnet or ferromagnetic material; wherein the electrode subassembly has a skin-facing surface and an outer perimeter; and one or more skin contact subassemblies each of which is configured to be removably coupled to the skin-facing surface of the electrode subassembly, wherein each skin contact subassembly of the one or more skin contact subassemblies comprises: an electrically conductive skin contact layer configured to contact skin of a subject; and at least one magnet or ferromagnetic material that is positioned so that the at least one magnet or ferromagnetic material of the skin contact subassembly is configured to magnetically attract the at least one magnet or ferromagnetic material of the electrode subassembly in a predetermined orientation between the electrode subassembly and said skin contact subassembly.

Aspect 17: The kit of aspect 16, wherein the electrode subassembly has a centroid, wherein the at least one magnet or ferromagnetic material is positioned radially outwardly from the centroid.

Aspect 18: The kit of aspect 17, wherein the at least one magnet or ferromagnetic material of the electrode subassembly comprises a plurality of magnets or ferromagnetic materials positioned radially outwardly from the centroid.

Aspect 19: A kit comprising: a netting or elasticated band/garment configured to be worn by a subject; an electrode subassembly comprising at least one electrode element and having a skin-facing surface; and at least one skin contact subassembly configured to removably couple to the skin-facing surface of the electrode subassembly to form an electrode assembly when coupled, wherein the skin contact subassembly comprises an electrically conductive skin contact layer configured to contact skin of the subject, wherein the netting or elasticated band/garment is configured to extend over the electrode assembly so that the electrode assembly is positioned between the netting or elasticated band/garment and the subject, wherein the netting or elasticated band/garment is configured to apply a pressure against the electrode assembly to secure the electrode assembly against the subject.

Aspect 20: The kit of aspect 19, wherein the netting or elasticated band/garment is configured to be worn on a torso of the subject, wherein the netting or elasticated band/garment includes at least one arm hole and a head opening.

Aspect 21: The kit of aspect 19, wherein the skin contact subassembly comprises an integral netting or elasticated band/garment, the integral netting or elasticated band/garment comprising one or more portions that extend outwardly from a periphery of the electrically conductive skin contact layer and fold around an outwardly facing surface of the electrode subassembly.

Aspect 22: A kit comprising: an electrode subassembly comprising at least one electrode element, wherein the electrode subassembly has a skin-facing surface, and at least one peripheral edge; at least one skin contact subassembly configured to be removably coupled to the skin-facing surface of the electrode subassembly, wherein one of the electrode subassembly or the at least one skin contact subassembly comprises at least one marking that is configured to align with an edge of the other of the electrode subassembly or the skin contact subassembly upon alignment between the electrode subassembly and the skin contact subassembly, wherein the at least one marking comprises a first color.

Aspect 23: The kit of aspect 22, wherein the at least one marking comprises a first color and a second color that is different than the first color.

Aspect 24: An assembly comprising: an electrode subassembly comprising at least one electrode element and having a skin-facing surface and an opposing outwardly facing surface; and a skin contact subassembly removably coupled to the skin-facing surface of the electrode subassembly, wherein the skin contact subassembly comprises: a base portion extending along a first axis and a second axis that is perpendicular to the first axis, wherein the base portion comprises an electrically conductive skin contact layer configured to contact skin of a subject; one or more flaps each having a respective edge congruent with or attached to the base portion and pivotably coupled to an edge of the base portion, wherein each flap of the one or more flaps is configured to pivot about its respective edge to attach to the outwardly facing surface of the electrode subassembly.

Aspect 25: The assembly of aspect 24, wherein the respective edge of each flap of the one or more flaps at least partially surrounds a receiving area of the base portion configured to receive at least a portion of the electrode subassembly thereagainst, wherein the electrode subassembly has an outer perimeter that overlies the receiving area and fits within an outer perimeter of the receiving area.

Aspect 26: The assembly of aspect 24, wherein the one or more flaps of the skin contact subassembly comprise at least two flaps.

Aspect 27: An assembly comprising: an electrode subassembly comprising at least one electrode element and having a skin-facing surface and an opposing outwardly facing surface; and a skin contact subassembly removably coupled to the skin-facing surface of the electrode subassembly, wherein the skin contact subassembly comprises: a base portion extending along a first axis and a second axis that is perpendicular to the first axis, wherein the base portion comprises an electrically conductive skin contact layer configured to contact skin of a subject; a plurality of flaps that pivotably couple to the base portion at respective edges, wherein the respective edges of the plurality of flaps cooperate to at least partially surround a receiving area of the base portion configured to receive at least a portion of the electrode subassembly thereagainst, wherein the electrode subassembly has an outer perimeter that overlies the receiving area.

Aspect 28: A kit comprising: an electrode subassembly comprising at least one electrode element having a skin-facing side, wherein the electrode subassembly has a skin-facing surface and at least one peripheral edge; a skin contact subassembly that is configured to be removably coupled to the skin-facing surface of the electrode subassembly, wherein the skin contact subassembly has a skin-facing surface and at least peripheral edge; a jig that is configured to contact portions of the peripheral edges of each of the electrode subassembly and the skin contact subassembly to orient the electrode subassembly and the skin contact subassembly relative to each other along a first axis and a second axis that is perpendicular to the first axis.

Aspect 29: The kit of aspect 28, wherein the electrode subassembly defines a first hole, wherein the at least one peripheral edge of the electrode subassembly comprises a first inner peripheral edge that surrounds the first hole of the electrode subassembly, wherein the skin contact subassembly defines a first hole, wherein the at least one peripheral edge of the skin contact subassembly comprises a first inner peripheral edge that surrounds the first hole of the skin contact subassembly, wherein the jig comprises: a base; and a first projection extending from the base, wherein the first projection configured to extend through the first holes of the electrode subassembly and the skin contact subassembly and contact the first inner peripheral edges of the first holes of the electrode subassembly and the skin contact subassembly.

Aspect 30: The kit of aspect 29, wherein the electrode subassembly defines a second hole, wherein the at least one peripheral edge of the electrode subassembly comprises a second inner peripheral edge that surrounds the second hole, wherein the skin contact subassembly defines a second hole, wherein the at least one peripheral edge of the skin contact subassembly comprises a second inner peripheral edge that surrounds the second hole of the skin contact subassembly, wherein the jig further comprises a second projection extending from the base, wherein the second projection is configured to extend through the second holes of the electrode subassembly and the skin contact subassembly and contact the second inner peripheral edges of the second holes of the electrode subassembly and the skin contact subassembly.

Aspect 31: The kit of aspect 28, wherein the at least one peripheral edge of the electrode subassembly comprises an outer peripheral edge, wherein the at least one peripheral edge of the skin contact subassembly comprises an outer peripheral edge, wherein the jig is configured to contact portions of the outer peripheral edges of each of the electrode subassembly and the skin contact subassembly to orient the electrode subassembly and the skin contact subassembly relative to each other.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

What is claimed is:

1. An assembly comprising:
an electrode subassembly comprising at least one electrode element and having a skin-facing surface and an opposing outwardly facing surface; and
a skin contact subassembly removably coupled to the skin-facing surface of the electrode subassembly, wherein the skin contact subassembly comprises:
a base portion extending along a first axis and a second axis that is perpendicular to the first axis, wherein the base portion comprises an electrically conductive skin contact layer configured to contact skin of a subject;
a plurality of flaps, each flap having a respective edge congruent with or attached to the base portion and pivotably coupled to the base portion, wherein the respective edges of the plurality of flaps cooperate to at least partially surround a receiving area of the base portion configured to receive at least a portion of the electrode subassembly thereagainst,
wherein the electrode subassembly has an outer perimeter that overlies the receiving area and fits within an outer perimeter of the receiving area.

2. The assembly of claim 1, wherein the plurality of flaps are configured to pivot about their respective edges to attach to the outwardly facing surface of the electrode subassembly.

3. The assembly of claim 2, wherein the plurality of flaps each comprise adhesive or hook or loop material that is configured to attach to the outwardly facing surface of the electrode subassembly.

4. The assembly of claim 2, wherein the electrode subassembly comprises adhesive or hook or loop material on the outwardly facing surface of the electrode subassembly, and wherein the plurality of flaps are configured to attach to the adhesive or hook or loop material on the outwardly facing surface of the electrode subassembly.

5. The assembly of claim 2, wherein the electrode subassembly comprises a magnet or ferromagnetic material on the outwardly facing surface of the electrode subassembly, and wherein the plurality of flaps each comprise a magnet or ferromagnetic material that is configured to attach to the magnet or ferromagnetic material on the outwardly facing surface of the electrode subassembly.

6. The assembly of claim 1, wherein the electrode subassembly has an operative conductive area, wherein the operative conductive area overlies the receiving area surrounded by the plurality of flaps of the skin contact subassembly.

7. The assembly of claim 1, wherein receipt of the outer perimeter of the electrode subassembly against the receiving area corresponds to proper alignment between the electrode subassembly and the skin contact subassembly.

8. The assembly of claim 1, wherein the plurality of flaps of the skin contact subassembly comprises at least three flaps.

9. A kit comprising:
an electrode subassembly comprising at least one electrode element and having a skin-facing surface, an opposing outwardly facing surface, and an outer perimeter; and
one or more skin contact subassemblies each of which is configured to be removably coupled to the skin-facing surface of the electrode subassembly, wherein each skin contact subassembly of the one or more skin contact subassemblies comprises:
a base portion extending along a first axis and a second axis that is perpendicular to the first axis, wherein the base portion comprises an electrically conductive skin contact layer configured to contact skin of a subject; and
a plurality of flaps each flap having a respective edge congruent with or attached to the base portion and pivotably coupled to the base portion, wherein the respective edges of the plurality of flaps cooperate to at least partially surround a receiving area of the base portion configured to receive at least a portion of the electrode subassembly thereagainst,
wherein each skin contact subassembly of the one or more skin contact subassemblies is configured to receive the electrode subassembly thereagainst so that an entirety of the outer perimeter of the electrode subassembly overlies the receiving area and fits within an outer perimeter of the receiving area.

10. The kit of claim 9, wherein the plurality of flaps of each skin contact subassembly of the one or more skin contact subassemblies are configured to pivot about their respective edges to attach to the outwardly facing surface of the electrode subassembly.

11. The kit of claim 10, wherein each flap of the plurality of flaps of each skin contact subassembly of the one or more skin contact subassemblies comprises adhesive or hook or loop material that is configured to attach to the outwardly facing surface of the electrode subassembly.

12. The kit of claim 10, wherein the electrode subassembly comprises adhesive or hook or loop material on the outwardly facing surface of the electrode subassembly, and wherein the plurality of flaps of each skin contact subassembly of the one or more skin contact subassemblies are configured to attach to the adhesive or hook or loop material on the outwardly facing surface of the electrode subassembly.

13. The kit of claim 10, wherein the electrode subassembly comprises a magnet or ferromagnetic material on the outwardly facing surface of the electrode subassembly, and wherein each flap of the plurality of flaps of each skin contact subassembly of the one or more skin contact subassemblies comprises a magnet or ferromagnetic material that is configured to attach to the magnet or ferromagnetic material on the outwardly facing surface of the electrode subassembly.

14. The kit of claim 9, wherein the electrode subassembly has an operative conductive area, wherein the operative conductive area is configured to overlie the receiving area surrounded by the plurality of flaps of a skin contact subassembly of the one or more skin contact subassemblies.

15. The kit of claim 9, wherein receipt of the outer perimeter of the electrode subassembly against the receiving area of a skin contact subassembly of the one or more skin contact subassemblies corresponds to proper alignment between the electrode subassembly and said skin contact subassembly.

16. The kit of claim 9, wherein the plurality of flaps of each skin contact subassembly of the one or more skin contact subassemblies comprises at least three flaps.

17. A method of using the kit of claim 9, the method comprising:
positioning the electrode subassembly against the receiving area of a skin contact subassembly of the one or more skin contact subassemblies so that the entirety of the outer perimeter of the electrode subassembly overlies the receiving area of the skin contact subassembly and fits within an outer perimeter of the receiving area; and
folding the flaps over the outwardly facing surface of the electrode subassembly.

18. An assembly comprising:
an electrode subassembly comprising at least one electrode element and having a skin-facing surface and an opposing outwardly facing surface; and
a skin contact subassembly removably coupled to the skin-facing surface of the electrode subassembly, wherein the skin contact subassembly comprises:
a base portion extending along a first axis and a second axis that is perpendicular to the first axis, wherein the base portion comprises an electrically conductive skin contact layer configured to contact skin of a subject;
one or more flaps each having a respective edge congruent with or attached to the base portion and pivotably coupled to an edge of the base portion, wherein each flap of the one or more flaps is configured to pivot about its respective edge to attach to the outwardly facing surface of the electrode subassembly.

19. The assembly of claim 18, wherein the respective edge of each flap of the one or more flaps at least partially surrounds a receiving area of the base portion configured to receive at least a portion of the electrode subassembly thereagainst, wherein the electrode subassembly has an outer perimeter that overlies the receiving area and fits within an outer perimeter of the receiving area.

20. The assembly of claim 18, wherein the one or more flaps of the skin contact subassembly comprise at least two flaps.
